(19) United States
(12) Patent Application Publication
(10) Pub. No.: US 2013/0084591 A1
(43) Pub. Date: Apr. 4, 2013

(54) ANALYTICAL TEST STRIP WITH ISOLATED BODILY FLUID PHASE-SHIFT AND ANALYTE DETERMINATION SAMPLE CHAMBERS

(71) Applicants: David McCOLL, Inverness (GB); Antony SMITH, Dingwall (GB); Lynsey WHYTE, Newtonmore (GB); Neil WHITEHEAD, Dingwall (GB); Ramsay DARLING, Inverness (GB)

(72) Inventors: David McCOLL, Inverness (GB); Antony SMITH, Dingwall (GB); Lynsey WHYTE, Newtonmore (GB); Neil WHITEHEAD, Dingwall (GB); Ramsay DARLING, Inverness (GB)

(73) Assignee: LifeScan Scotland Ltd., Inverness-shire (GB)

(21) Appl. No.: 13/250,747

(22) Filed: Sep. 30, 2011

(51) Int. Cl. C12Q 1/02; C12M 1/34

(52) U.S. Cl. 435/29; 435/287.9

(57) ABSTRACT

An analytical test strip (“ATS”) for use with a hand-held test meter in the determination of an analyte in a bodily fluid sample includes an electrically insulting substrate, a first patterned conductor layer disposed on the electrically insulating substrate and having a working electrode and a reference electrode. The ATS also includes an enzymatic reagent layer disposed on the working electrode, a first patterned spacer layer disposed over the first patterned conductor layer and defining both a first sample-receiving channel and an analyte determination sample chamber within the ATS, and a second patterned spacer layer disposed over the first patterned spacer layer and defining at least a second sample-receiving channel. The ATS further includes a bodily fluid phase-shift sample chamber in fluidic communication with the second sample-receiving channel. The first sample-receiving channel and analyte determination sample chamber are isolated from the second sample-receiving channel and bodily fluid phase-shift sample chamber.

### BACKGROUND OF THE INVENTION

1. Field of the Invention

The present invention relates, in general, to medical devices and, in particular, to analytical test strips and related methods.

2. Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using a hand-held test meter in combination with analytical test strips (e.g., electrochemical-based analytical test strips).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified, perspective exploded view of an analytical test trip according to an embodiment of the present invention;

FIG. 2A is a simplified top view of the electrically-insulating substrate and a portion of a first patterned conductor layer of an analytical test strip of FIG. 1;

FIG. 2B is a simplified top view of the first patterned spacer layer of the analytical test strip of FIG. 1;

FIG. 2C is a simplified top view of the second patterned spacer layer of the analytical test strip of FIG. 1;

FIG. 3 is a simplified cross-sectional side view of the analytical test strip of FIG. 1 taken along line A-A of FIG. 2A;

FIG. 4 is a simplified, perspective exploded view of an analytical test trip according to another embodiment of the present invention;

FIG. 5A is a simplified top view of the electrically insulating substrate and first patterned conductor layer of the analytical test strip of FIG. 4;

FIG. 5B is a simplified top view of a portion of a second patterned spacer layer and second patterned conductor layer of the analytical test strip of FIG. 4;

FIG. 5C is a simplified top view of a third patterned spacer layer of the analytical test strip of FIG. 4;

FIG. 6 is a simplified cross-sectional side view of the analytical test strip of FIG. 4 taken along line B-B of FIG. 5A; and

FIG. 7 is a flow diagram depicting stages in a method for determining and analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, analytical test strips (e.g., an electrochemical-based analytical test strip) for use with a hand-held test meter in the determination of an analyte (such as glucose) in a bodily fluid sample (for example, a whole blood sample) include an electrically insulting substrate, a first patterned conductor layer disposed on the electrically insulating substrate and having a working electrode and a reference electrode. The analytical test strip also includes an enzymatic reagent layer disposed on the working electrode, a first patterned spacer layer disposed over the first patterned conductor layer and defining both a first sample-receiving channel and an analyte determination sample chamber within the analytical test strip, and a second patterned spacer layer disposed over the first patterned spacer layer and defining at least a second sample-receiving channel. In addition, the analytical test strip further includes a bodily fluid phase-shift sample chamber in fluidic communication with the second sample-receiving channel. Moreover, the first sample-receiving channel and analyte determination sample chamber of the analytical test strip are isolated from the second sample-receiving channel and bodily fluid phase-shift sample chamber of the analytical test strip.

Analytical test strips according to embodiments of the present invention are beneficial in that, for example, the isolation (fluidic and electrical) between the analyte determination sample chamber and the bodily fluid phase-shift sample chamber prevents potential interference between the determination of the analyte in the bodily fluid sample and a phase-shift measurement of the bodily fluid. Analytical test strips according to some embodiments of the present invention are also beneficial in that the first sample-receiving channel and analyte determination chamber are separated from the second sample-receiving channel and bodily fluid phase-shift sample chamber by portions of the first and/or second patterned spacer layers that can be beneficially thin, thus providing for an analytical test strip with a small, yet mechanically stable, cross-section.

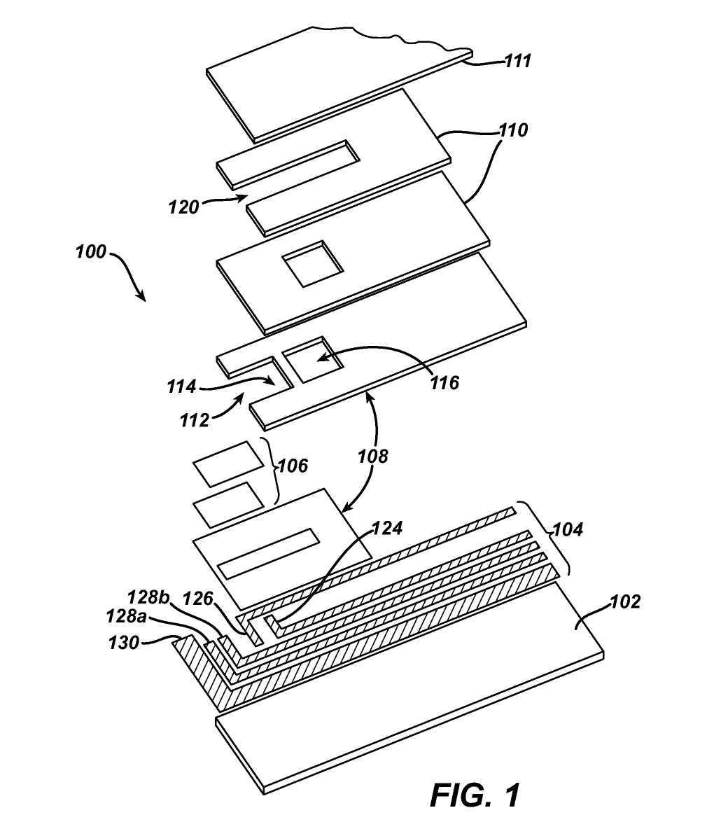

Referring to FIGS. 1, 2A-2C and 3, electrochemical-based analytical test strip 100 includes an electrically-insulating substrate 102, a first patterned conductor layer 104 disposed on the electrically-insulating substrate layer, an enzymatic reagent layer 106 (for clarity depicted in FIG. 1 only), a first patterned spacer layer 108, a second patterned spacer layer 110, and a top cover 111. In the embodiment of FIG. 1, first pattered spacer layer 108 and second patterned spacer layer 110 are depicted as bi-layer structures. However, once apprised of the present disclosure one skilled in the art will recognize that first and second patterned spacer layers employed in embodiments of the present invention can be unitary layers or any other suitably formatted layer.

First patterned spacer layer 108 is configured such that electrochemical-based analytical test strip 100 also includes a first sample-receiving channel 112 and an analyte determination sample chamber 114. First patterned spacer layer 108 is also configured to define a bodily fluid phase-shift sample chamber 116 and an analyte determination sample chamber vent 118 (for clarity not depicted in FIG. 1).

Second patterned spacer layer 110 is configured to define a second sample-receiving channel 120 and a bodily fluid phase-shift chamber vent 122 (for clarity not depicted in FIG. 1).

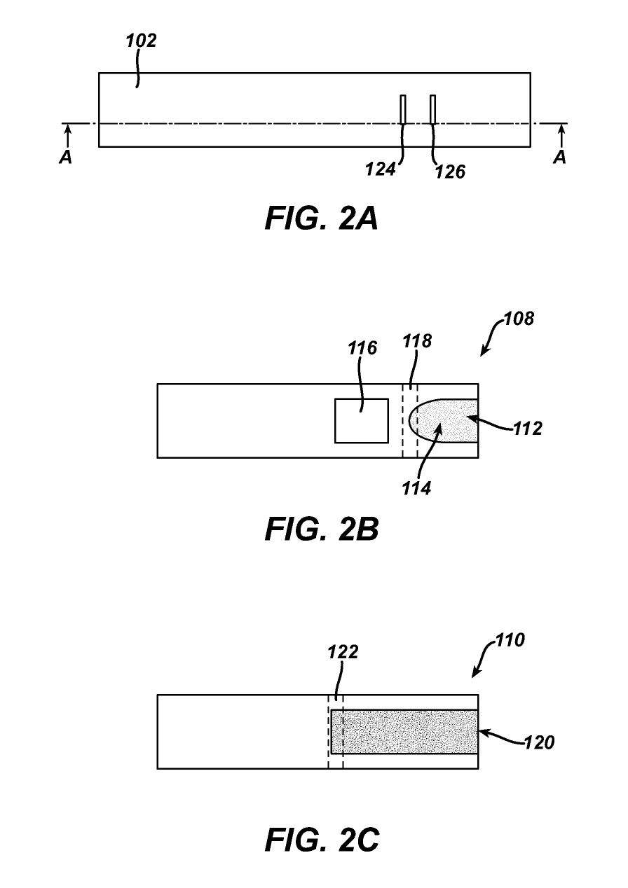

First patterned conductor layer 104 includes a first phase-shift measurement electrode 124, a second phase-shift measurement electrode 126, two working electrodes 128a and 128b and a reference electrode 130. For clarity, FIG. 2A depicts only first phase-shift measurement electrode 124 and second phase-shift measurement electrode 126 and not the entirety of first patterned conductor layer 104.

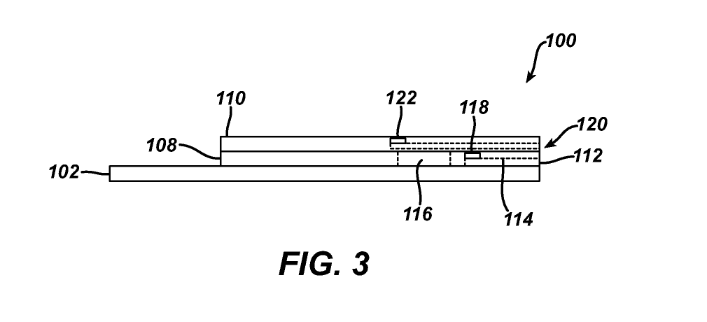

First sample-receiving channel 112 and analyte determination sample chamber 114 are isolated, both fluidically and electrically, from second sample-receiving channel 120 and bodily fluid phase-shift sample chamber 116 (see FIG. 3 in particular wherein the first and second patterned conductor layers are omitted for clarity). Moreover, in the embodiment of FIG. 3, the bodily fluid phase-shift sample chamber is disposed in a side-by-side configuration with the analyte determination sample chamber.

During use of electrochemical-based analytical test strip 100 to determine an analyte in a bodily fluid sample (e.g., blood glucose concentration in a whole blood sample), working and reference electrodes are employed by an associated meter (not shown) to monitor an electrochemical response of the electrochemical-based analytical test strip. The electrochemical response can be, for example, an electrochemical reaction induced current of interest. The magnitude of such a current can then be correlated, taking into consideration the haematocrit of the bodily fluid sample as determined by the bodily fluid sample's phase shift, with the amount of analyte present in the bodily fluid sample under investigation. During such use, a bodily fluid sample is applied to electrochemical-based analytical test strip 100 and, thereby, received in both analyte determination sample chamber 114 and bodily fluid phase-shift sample chamber 116.

Electrically-insulating substrate 102 can be any suitable electrically-insulating substrate known to one skilled in the art including, for example, a nylon substrate, polycarbonate substrate, a polyimide substrate, a polyvinyl chloride substrate, a polyethylene substrate, a polypropylene substrate, a glycolated polyester (PETG) substrate, a polystyrene substrate, a silicon substrate, ceramic substrate, glass substrate or a polyester substrate (e.g., a 7 mil thick polyester substrate). The electrically-insulating substrate can have any suitable dimensions including, for example, a width dimension of about 5 mm, a length dimension of about 27 mm and a thickness dimension of about 0.5 mm.

First patterned conductor layer 104 can be formed of any suitable electrically conductive material such as, for example, gold, palladium, carbon, silver, platinum, tin oxide, iridium, indium, or combinations thereof (e.g., indium doped tin oxide). Moreover, any suitable technique or combination of techniques can be employed to form first patterned conductor layer 104 including, for example, sputtering, evaporation, electro-less plating, screen-printing, contact printing, laser ablation or gravure printing. A typical but non-limiting thickness for the patterned conductor layer is in the range of 5 nm to 100 nm.

One skilled in the art will recognize that conventional electrochemical-based analyte test strips employ a working electrode along with an associated counter/reference electrode and enzymatic reagent layer to facilitate an electrochemical reaction with an analyte of interest and, thereby, determine the presence and/or concentration of that analyte. For example, an electrochemical-based analyte test strip for the determination of glucose concentration in a blood sample can employ an enzymatic reagent that includes the enzyme glucose oxidase and the mediator ferricyanide (which is reduced to the mediator ferrocyanide during the electrochemical reaction). Such conventional analyte test strips and enzymatic reagent layers are described in, for example, U.S. Pat. Nos. 5,708,247; 5,951,836; 6,241,862; and 6,284,125; each of which is hereby incorporated in full by reference. In this regard, the reagent layer employed in embodiments of the present invention can include any suitable sample-soluble enzymatic reagents, with the selection of enzymatic reagents being dependent on the analyte to be determined and the bodily fluid sample. For example, if glucose is to be determined in a blood sample, enzymatic reagent layer 106 can include glucose oxidase or glucose dehydrogenase along with other components necessary for functional operation.

In general, enzymatic reagent layer 106 includes at least an enzyme and a mediator. Examples of suitable mediators include, for example, ferricyanide, ferrocene, ferrocene derivatives, osmium bipyridyl complexes, and quinone derivatives. Examples of suitable enzymes include glucose oxidase, glucose dehydrogenase (GDH) using a pyrroloquinoline quinone (PQQ) co-factor, GDH using a nicotinamide adenine dinucleotide (NAD) co-factor, and GDH using a flavin adenine dinucleotide (FAD) co-factor. Enzymatic reagent layer 106 can be applied during manufacturing using any suitable technique including, for example, screen printing.

Once apprised of the present disclosure, one skilled in the art will recognize that enzymatic reagent layer 106 can, if desired, also contain suitable buffers (such as, for example, Tris HCl, Citraconate, Citrate and Phosphate), hydroxyethylcelulose [NEC], carboxymethylcellulose, ethycellulose and alginate, enzyme stabilizers and other additives as are known in the field.

Further details regarding the use of electrodes and enzymatic reagent layers for the determination of the concentrations of analytes in a bodily fluid sample, albeit in the absence of the phase-shift measurement electrodes, bodily-fluid phase-shift sample chambers and second sample receiving channels analytical test strips and related methods described herein, are in U.S. Pat. No. 6,733,655, which is hereby fully incorporated by reference.

First and second patterned spacer layers 108 and 110 respectively can be formed of any suitable material including, for example, a 95 um thick, double-sided pressure sensitive adhesive layer, a heat activated adhesive layer, or a thermo-setting adhesive plastic layer. First patterned spacer layer 108 can have, for example, a thickness in the range of from about 1 micron to about 500 microns, preferably between about 10 microns and about 400 microns, and more preferably between about 40 microns and about 200 microns.

Electrochemical-based analytical test strip 100 can be manufactured, for example, by the sequential aligned formation of first patterned conductor layer 104, enzymatic reagent layer 106, first patterned spacer layer 108, and second patterned spacer layer 110 onto electrically-insulating substrate 102. Any suitable techniques known to one skilled in the art can be used to accomplish such sequential aligned formation, including, for example, screen printing, photolithography, photogravure, chemical vapour deposition, sputtering, tape lamination techniques and combinations thereof.

Analytical test strops according to embodiments can be configured, for example, for operable electrical connection and use with the analytical test strip sample cell interface of a hand-held test meter as described in co-pending patent application Ser. No. 13/250,525 [tentatively identified by attorney docket number DDI5209USNP], which is hereby incorporated in full be reference.

It has been determined that a relationship exists between the reactance of a whole blood sample and the hematocrit of that sample. Electrical modeling of a bodily fluid sample (i.e., a whole blood sample) as parallel capacitive and resistive components indicates that when an alternating current (AC) signal is forced through the bodily fluid sample, the phase shift of the AC signal will be dependent on both the frequency of the AC voltage and the hematocrit of the sample. Therefore, the hematocrit of a bodily fluid sample can be measured by, for example, driving AC signals of known frequency through the bodily fluid sample and detecting their phase shift. The phase-shift measurement electrodes of analytical test strips according to embodiments of the present invention are particularly suitable for use in such phase-shifty measurements since the first and second phase shift measurement electrodes are in direct contact with a bodily fluid sample present in the sample chamber. Moreover, a bodily fluid sample hematocrit ascertained from a phase shift measurement(s) can be employed to compensate for the effect of hematocrit during analyte determination.

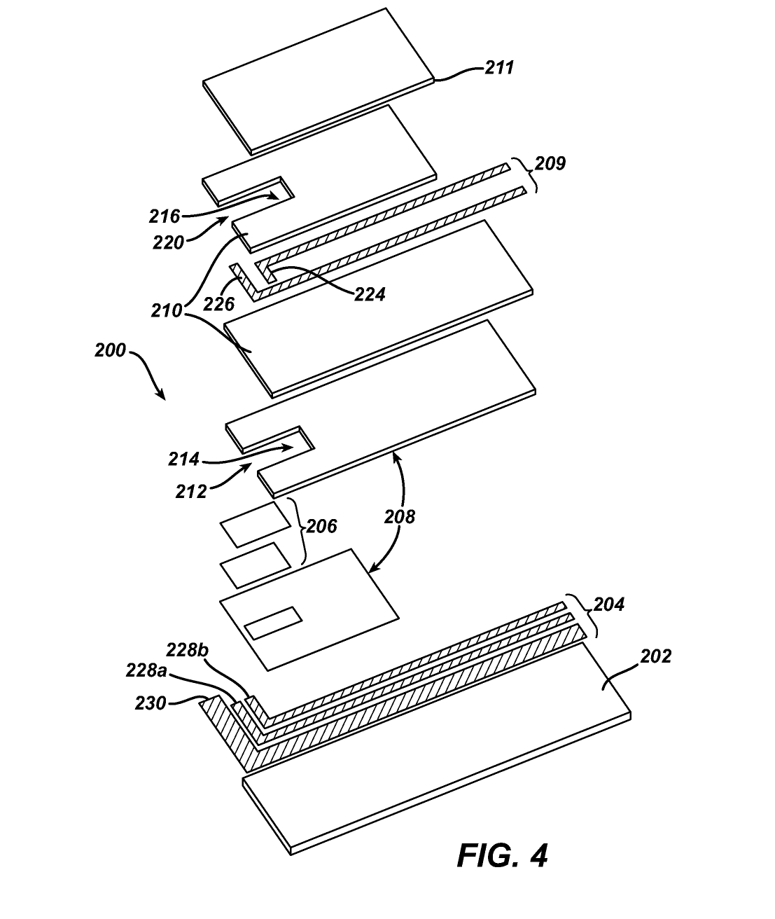

Referring to FIGS. 4, 5A-5C and 6, electrochemical-based analytical test strip 200 includes an electrically-insulating substrate 202, a first patterned conductor layer 204 disposed on the electrically-insulating substrate layer, an enzymatic reagent layer 206 (for clarity depicted in FIG. 4 only), a first patterned spacer layer 208, a second patterned conductor layer 209, a second patterned spacer layer 210, and a top cover 211. In the embodiment of FIG. 4, first pattered spacer layer 208 and second patterned spacer layer 210 are depicted as bi-layer structures. However, once apprised of the present disclosure one of skill in the art will recognize that first and second patterned spacer layers employed in embodiments of the present invention can be unitary layers or any other suitably formatted layer.

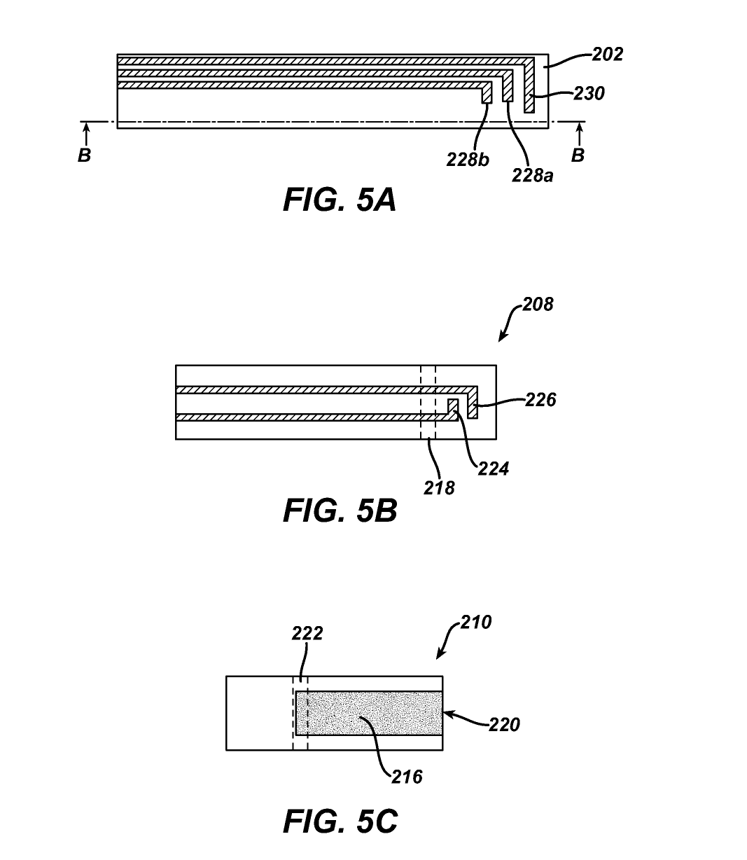

First patterned spacer layer 208 is configured such that electrochemical-based analytical test strip 200 also includes a first sample-receiving channel 212, an analyte determination sample chamber 214 and an analyte determination sample chamber vent 218 (not depicted in FIG. 4 but depicted with dashed lines in FIG. 5B). Analyte determination sample chamber vent 218 is configured to aid in the introduction of a bodily fluid sample into analyte determination sample chamber 214 via first sample-receiving channel 212.

Second patterned spacer layer 210 is configured to define a second sample-receiving channel 220, a bodily fluid phase-shift sample chamber 216 and a bodily fluid phase-shift chamber vent 222 (not depicted in FIG. 4 but depicted with dashed lines in FIG. 5C). Bodily fluid phase-shift chamber vent 222 is configured to aid in the introduction of a bodily fluid sample into bodily fluid phase-shift sample chamber 216 via second sample-receiving channel 220.

First patterned conductor layer 204 two working electrodes 228a and 228b (depicted in FIGS. 4 and 5A) and a reference electrode 230 (also depicted in FIGS. 4 and 5A). Second patterned conductor layer 209 includes a first phase-shift measurement electrode 224 and a second phase-shift measurement electrode 226 and is disposed above first patterned spacer layer 208 and embedded in the bi-layer structure of second pattered spacer layer 210.

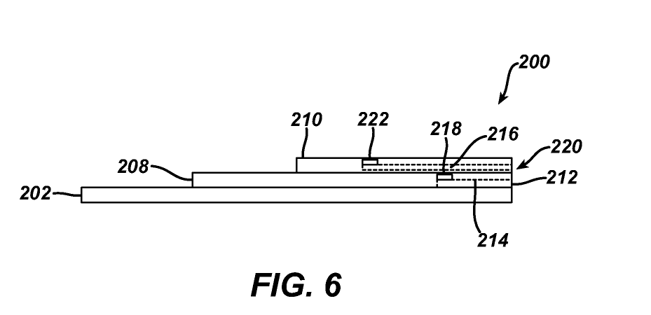

First sample-receiving channel 212 and analyte determination sample chamber 214 are isolated, both fluidically and electrically, from second sample-receiving channel 220 and bodily fluid phase-shift sample chamber 216 (see FIG. 6 in particular wherein the first and second patterned conductor layers are not depicted for clarity).

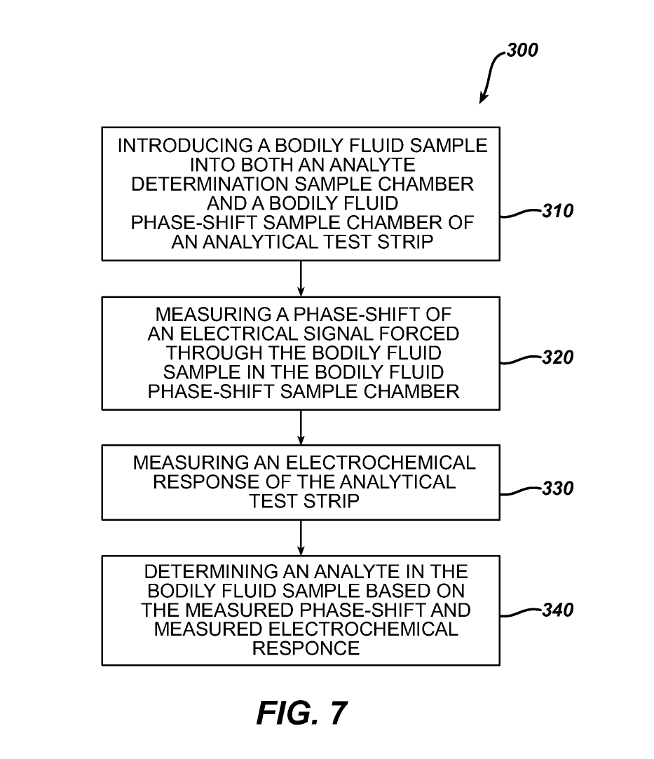

FIG. 7 is a flow diagram depicting stages in a method 300 for determining and analyte (such as glucose) in a bodily fluid sample (for example, a whole blood sample) according to an embodiment of the present invention.

Method 300 includes introducing a bodily fluid sample into both an analyte determination sample chamber and a bodily fluid phase-shift sample chamber of an analytical test strip (see step 310 of FIG. 7). In such an introduction step, the analyte determination sample chamber has disposed therein at least one working electrode and a reference electrode. Moreover, the bodily fluid phase-shift sample chamber has disposed therein a first phase-shift measurement electrode and a second phase-shift measurement electrode.

At step 320 of method 300, measuring a phase shift of an electrical signal forced through the bodily fluid sample in the bodily fluid phase-shift sample chamber via the first phase-shift measurement electrode and the second phase-shift measurement electrode is measured. The bodily fluid phase-shift sample chamber is isolated (both fluidic and electrically) from the analyte determination sample chamber to prevent deleterious interference between the phase-shift and electrochemical response measurements.

Method 300 also includes measuring an electrochemical response of the analytical test strip using the at least one working electrode and reference electrode (see step 330) and determining an analyte in the bodily fluid sample based on the measured phase shift and the measured electrochemical response (see step 340).

Once apprised of the present disclosure, one skilled in the art will recognize that methods according to embodiments of the present invention, including method 300, can be readily modified to incorporate any of the techniques, benefits and characteristics of analytical test strips according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. An analytical test strip for use with a hand-held test meter in the determination of an analyte in a bodily fluid sample, the analytical test strip comprising:
an electrically insulting substrate;
a first patterned conductor layer disposed on the electrically insulating substrate, the first patterned conductor layer including at least:
a working electrode; and
a reference electrode;
an enzymatic reagent layer disposed on at least the working electrode;
a first patterned spacer layer disposed over the first patterned conductor layer and defining a first sample-receiving channel and an analyte determination sample chamber within the analytical test strip;
a second patterned spacer layer disposed over the first patterned spacer layer and defining at least a second sample-receiving channel; and
a bodily fluid phase-shift sample chamber in fluidic communication with the second sample-receiving channel,
wherein the first sample-receiving channel and analyte determination sample chamber are isolated from the second sample-receiving channel and bodily fluid phase-shift sample chamber.

2. The analytical test strip of claim 1 further including:
a first phase-shift measurement electrode disposed in the bodily fluid phase-shift sample chamber; and
a second phase-shift measurement electrode disposed in the bodily fluid phase-shift sample chamber; and
wherein the working electrode and reference electrode are disposed in the analyte determination sample chamber,

3. The analytical test strip of claim 2 wherein the second patterned spacer layer defines the bodily fluid phase-shift sample chamber.

4. The analytical test strip of claim 3 further including a second patterned conductor layer disposed over the first patterned spacer layer and including the first phase-shift measurement electrode disposed in the bodily fluid phase-shift sample chamber and the second phase-shift measurement electrode disposed in the bodily fluid phase-shift sample chamber

5. The analytical test strip of claim 2 wherein the first patterned spacer layer defines the bodily-fluid phase-shift sample chamber.

6. The analytical test strip of claim 5 wherein the first patterned conductor layer further includes:
a first phase-shift measurement electrode disposed in the bodily fluid phase-shift sample chamber; and
a second phase-shift measurement electrode disposed in the bodily fluid phase-shift sample chamber.

7. The analytical test strip of claim 2 wherein the first phase-shift measurement electrode and the second phase-shift measurement electrode are configured to force an electrical signal through the bodily fluid sample in the sample chamber.

8. The analytical test strip of claim 2 wherein the first phase-shift measurement electrode and the second phase-shift measurement electrode are configured to force an electrical signal of known frequency through the bodily fluid sample in the sample chamber.

9. The analytical test strip of claim 1 wherein the analytical test strip is an electrochemical-based analytical test strip configured for the determination of glucose in a whole blood sample.

10. The analytical test strip of claim 1 wherein the first patterned spacer layer further defines an analyte determination sample chamber vent.

11. The analytical test strip of claim 1 wherein the second patterned spacer layer further defines a bodily fluid phase-shift sample chamber vent.

12. A method for determining an analyte in a bodily fluid sample, the method comprising:
introducing a bodily fluid sample into both an analyte determination sample chamber and a bodily fluid phase-shift sample chamber of an analytical test strip, the analyte determination sample chamber having disposed therein:
at least one working electrode; and
a reference electrode;
and the bodily fluid phase-shift sample chamber having disposed therein:
a first phase-shift measurement electrode; and
a second phase-shift measurement electrode;
measuring a phase shift of an electrical signal forced through the bodily fluid sample in the bodily fluid phase-shift sample chamber via the first phase-shift measurement electrode and the second phase-shift measurement electrode;
measuring an electrochemical response of the analytical test strip using the at least one working electrode and reference electrode; and
determining an analyte in the bodily fluid sample based on the measured phase shift and the measured electrochemical response,
wherein the analyte determination sample chamber is isolated from the bodily fluid phase-shift sample chamber.

13. The method of claim 12 wherein the analyte is glucose and the bodily fluid sample is a whole blood sample.

14. The method of claim 12 wherein the bodily fluid phase-shift sample chamber is disposed over the analyte determination sample chamber.

15. The method of claim 12 wherein the analytical test strip includes a first sample receiving channel in fluidic communication with the analyte determination sample chamber and a second sample receiving channel in fluidic communication with the bodily fluid phase-shift sample chamber.

16. The method of claim 15 wherein the second sample receiving channel is disposed over the first sample receiving chamber.

17. The method of claim 16 wherein the bodily fluid phase-shift sample chamber is disposed in a side-by-side configuration with the analyte determination sample chamber.

18. The method of claim 16 wherein the bodily fluid phase-shift sample chamber is disposed over the analyte determination sample chamber.

19. The method of claim 12 wherein the measuring of the electrochemical response employs the working electrode and the reference electrode.

20. The method of claim 12 wherein the bodily fluid sample is introduced into the analyte determination sample chamber and the bodily fluid-phase shift sample chamber aided by an analyte determination sample chamber vent of the analytical test strip and a bodily fluid phase-shift sample chamber vent of the analytical test strip.

21. The method of claim 12 wherein the measuring of the phase shift and the measuring of an electrochemical response is accomplished with a hand-held test meter.

22. The method of claim 12 wherein the determining step employs the measured phase shift to ascertain the hematocrit of the bodily fluid sample and the ascertained hematocrit is employed in the determining of the analyte.
